(19) United States
(12) Patent Application Publication
(10) Pub. No.: US 2004/0038352 A1
(43) Pub. Date: Feb. 26, 2004

(54) Method for fermentative production of amino acids and amino acid derivatives of the phosphoglycerate family

(75) Inventor: Thomas Maier, Dachau (DE)

Correspondence Address: COLLARD & ROE, P.C., 1077 Northern Boulevard, Roslyn, NY, 11576, US

(73) Assignee: CONSORTIUM FUR ELEKTROCHEMISCHE INDUSTRIE GmbH

(21) Appl. No.: 10/620,487

(22) Filed: Jul. 16, 2003

(30) Foreign Application Priority Data
Jul. 19, 2002 (DE) 102 32 930.3

(51) Int. Cl. C12P 13/04; C12N 1/21

(52) U.S. Cl. 435/106; 435/252.33

(57) ABSTRACT

A microorganism strain suitable for fermentative production of amino acids of the phosphoglycerate family or derivatives thereof and producible from a starting strain, is based upon a starting strain having an increased activity of a yfiK-gene product or of a gene product of a yfiK homologue.

### BACKGROUND OF THE INVENTION

1. Field of the Invention

The invention relates to a method for producing amino acids and amino acid derivatives of the phosphoglycerate family such as, for example, O-acetyl-L-serine, N-acetyl-L-serine, L-cysteine, LL-cystine and L-cysteine derivatives by means of fermentation.

2. The Prior Art

The twenty natural proteinogenic amino acids are usually produced these days via fermentation of microorganisms. Here, use is made of the fact that microorganisms possess appropriate biosynthetic pathways for synthesis of said natural amino acids.

However, such biosynthetic pathways are strictly regulated in wild-type strains, ensuring that the cell produces said amino acids only for its own needs. An important precondition for efficient production processes is therefore to have suitable microorganisms available whose performance of producing the desired amino acid is drastically increased, in contrast to wild-type organisms.

Such amino acid-overproducing microorganisms can be generated by means of classical mutation/selection methods and/or modern specific recombinant techniques (“metabolic engineering”). The latter first involves the identification of genes or alleles which lead to overproduction, due to their modification, activation or inactivation. These genes/alleles are then, by means of molecular-biological techniques, introduced into a microorganism strain or inactivated so as to achieve optimal overproduction. Frequently, however, only the combination of a plurality of different measures results in a truly efficient production.

The phosphoglycerate family of amino acids are defined by the fact that they are biosynthetically derived from 3-phosphoglyceric acid. The natural metabolic pathway leads initially via the intermediates 3-phosphohydroxypyruvate and 3-phospho-L-serine to L-serine. L-serine can be converted further to glycine or, via o-acetyl-L-serine, to L-cysteine.

Some genes/alleles for fermentative production of amino acids of the phosphoglycerate family, in particular of L-serine and L-cysteine, whose use results in amino acid overproduction are already known in the prior art:

serA-alleles, as described in EP0620853B1 or EP0931 833A2.

These serA alleles code for 3-phosphoglycerate dehydro genases which are subject to a reduced feedback inhibition by L-serine. This substantially decouples the formation of 3-hydroxypyruvate from the cellular serine level.

cysE alleles, as described in WO 97/15673 (hereby incorporated by reference) or

Nakamori S. et al., 1998, Appl. Env. Microbiol. 64: 1607-1611 (hereby incorporated by reference) or

Takagi H. et al., 1999, FEBS Lett. 452: 323-327, which are introduced into a microorganism strain. These cysE alleles code for serine O-acetyl transfer as which are subject to a reduced feedback inhibition by L-cysteine. This substantially decouples the formation of O-acetyl-L-serine or L-cysteine from the cellular cysteine level.

Efflux genes are described in EP0885962A1. The orf gene described presumably codes for an efflux system suitable for exporting antibiotics and other toxic substances and resulting in overproduction of L-cysteine, L-cystine, N-acetyl-serine and/or thiazolidine derivatives.

CysB gene, is described in DE19949579C1. The cysB gene codes for a central gene regulator of sulfur metabolism and thus plays a decisive part in providing sulfide for cysteine biosynthesis.

It is likewise known from the prior art that the methods stated can also lead to cysteine derivatives. Thus, LL-cystine can be formed as an oxidation product from L-cysteine or 2-methylthiazolidine-2,4-dicarboxylic acid can be formed as condensation product from L-cysteine and pyruvate during fermentation. Since L-cysteine is the central sulfur donor of the cell, it is also possible to use the methods described as a starting point for producing a large variety of sulfur-containing metabolites (e.g. L-methionine, (+)-biotin, thiamine, etc.) which, in accordance with the present invention, are to be regarded as L-cysteine derivatives.

The fact that it is also possible to produce, using a suitable procedure, the amino acids N-acetyl-L-serine (EP-A1-0885962) and O-acetyl-L-serine (DE-A-10107002) as main fermentation products has also been described. According to DE-A-10219851, L-serine can in turn be recovered relatively easily from N-acetyl-L-serine-containing fermentation broth.

### SUMMARY OF THE INVENTION

It is an object of the present invention to provide a recombinant microorganism strain which enables amino acids or amino acid derivatives of the phosphoglycerate family to be overproduced. Another object is to provide a fermentative method for producing amino acids or amino acid derivatives of the phosphoglycerate family by means of said recombinant microorganism strain.

The above object is achieved by a microorganism strain suitable for fermentative production of amino acids of the phosphoglycerate family or derivatives thereof and producible from a starting strain, in which the activity of the yfiK-gene product or of a gene product of a yfik homologue is increased compared to said starting strain.

In accordance with the present invention, the activity of the yfiK-gene product is also increased when, due to an increase in the amount of gene product in the cell, the overall activity in the cell is increased and thus the activity of the yfiK-gene product per cell, although the specific activity of said gene product remains unchanged.

As part of the sequencing of the Escherichia coli genome (Blattner et al. 1997, Science 277:1453-1462) the yfiK gene was identified as open reading frame and codes for a protein with 195 amino acids. Up until now it has not been possible to assign a physiological function to the yfiK gene. A database search for proteins with sequence homology (FASTA algorithm of the GCG Wisconsin Package, Genetics Computer Group (GLG) Madison, Wis.) is also not very conclusive, since only similarities to proteins whose function is likewise unknown are indicated. The only clue for a possible activity of the yfiK-gene product can be found in Aleshin et al. (Trends in Biol. Sci., 1999, 24: 133-135). The authors of this publication postulate a structural motive which should characterize a protein family of amino acid-efflux proteins. Since this weak consensus motif also occurs in the YfiK protein, the latter could be an efflux system for amino acids. However, it is absolutely impossible for the skilled worker to draw conclusions therefrom about concrete amino acid substrates of said YfiK protein. The finding that the YfiK gene product contributes favorably to the production of amino acids of the phosphoglycerate family is surprising, in particular since an efflux protein for amino acids of the phosphoglycerate family in Escherichia coli, namely the YdeD gene product, has already been characterized (DaBler et al. Mol. Microbiol., 2000, 36: 1101-1112) and the existence of a second system is completely unexpected. Interestingly, there exist no structural similarities between the yfiK- and ydeD-gene products.

The yfiK gene and the YfiK gene product (YfiK protein) are characterized by the sequences SEQ ID No. 1 and SEQ ID No. 2, respectively. Within the scope of the present invention, those genes whose sequence identity in an analysis using the BESTFIT algorithm (GCG Wisconsin Package, Genetics Computer Group (GLG) Madison, Wis.) is more than 30% are to be regarded as yfik homologues. Particular preference is given to a sequence identity of more than 70%.

Likewise, proteins having a sequence identity of more than 30% (BESTFIT algorithm (GCG Wisconsin Package, Genetics Computer Group (GLG) Madison, Wis.) are to be regarded as YfiK homologous proteins. Particular preference is given to a sequence identity of more than 70%.

Thus, yfiK homologues mean also allele variants of the yfiK gene, in particular functional variants, which are derived from the sequence depicted in SEQ ID No. 1 by deletion, insertion or substitution of nucleotides, with the enzymic activity of the respective gene product being retained, however.

Microorganisms of the invention which have an increased activity of the yfik-gene product compared to the starting strain can be generated using standard techniques of molecular biology.

Suitable starting strains are in principle any organisms which have the biosynthetic pathway for amino acids of the phosphoglycerate family, are accessible to recombinant methods and can be cultured by fermentation. Microorganisms of this kind may be fungi, yeasts or bacteria. They are preferably bacteria of the phylogenetic group of eubacteria and particularly preferably microorganisms of the family Enterobacteriaceae, and in particular of the species Escherichia coli.

The activity of the yfiK-gene product in the microorganisms of the invention is increased, for example, by increasing expression of the yfiK gene. It is possible to increase the copy number of the yfiK gene in a microorganism and/or to increase expression of the yfiK gene by means of suitable promoters. Increased expression means preferably that expression of the yfiK gene is at least twice as high as in the starting strain.

The copy number of the yfiK gene in a microorganism can be increased using methods known to the skilled worker. Thus it is possible; for example, to clone the yfiK gene into plasmid vectors having multiple copies per cell (e.g. pUC19, pBR322, pACYC184 for Escherichia coli) and to introduce it in this way into said microorganism. Alternatively, multiple copies of the yfiK gene may be integrated into the chromosome of a microorganism. Integration methods which may be used are the known systems using temperate bacteriophages, integrative plasmids or integration via homologous recombination (e.g. Hamilton et al., 1989, J. Bacteriol. 171: 4617-4622).

Preference is given to increasing the copy number by cloning a yfiK gene into plasmid vectors under the control of a promoter. Particular preference is given to increasing the copy number in Escherichia coli by cloning a yfiK gene into a pACYC derivative such as, for example, pACYC184-LH (deposited, in accordance with the Budapest Treaty, with the Deutsche Sammlung fur Mikroorganismen und Zellkulturen, Braunschweig, Germany on 8.18.95 under the number DSM 10172). in accordance with the Budapest Treaty, with the Deutsche Sammlung fur Mikroorganismen und Zellkulturen, Braunschweig, Germany on 8.18.95 under the number DSM 10172).

The natural promoter and operator region of the gene may serve as control region for expressing a plasmid-encoded yfiK gene.

In particular, however, expression of a yfik gene may also be increased by means of other promoters. Appropriate promoter systems such as, for example, the constitutive GAPDH promoter of the gapA gene or the inducible lac, tac, trc, lambda, ara or tet promoters in Escherichia coli are known to the skilled worker (Makrides S. C., 1996, Microbiol. Rev. 60: 512-538). Such constructs may be used in a manner known per se on plasmids or chromosomally.

It is furthermore possible to increase the expression by the particular construct containing translational starter signals such as, for example, the ribosomal binding site or the start codon of the gene in optimized sequence or by replacing codons which are rare according to the “codon usage” by codons occurring more frequently.

Microorganism strains having the modifications mentioned are preferred embodiments of the present invention.

A yfiK gene is cloned into plasmid vectors, for example, by specific amplification by means of the polymerase chain reaction using specific primers which cover the complete yfiK gene and subsequent ligation with vector-DNA fragments.

Preferred vectors used for cloning a yfiK gene are plasmids which already contain promoters for increased expression, for example the constitutive GAPDH promoter of the Escherichia coli gapA gene.

The invention thus also relates to a plasmid which comprises a yfiK gene having a promoter.

Particular preference is furthermore given to vectors which already contain a gene/allele whose use results in overproduction of amino acids of the phosphoglycerate family, such as, for example, the cysEX gene (W097/15673). Such vectors make it possible to prepare inventive microorganism strains with high amino acid overproduction directly from any microorganism strain, since such a plasmid also reduces the feedback inhibition of cysteine metabolism in a microorganism.

The invention thus also relates to a plasmid which comprises a genetic element for the deregulatuion of cycsteine metabolism and a yfiK gene with a promoter.

A common transformation method (e.g. electroporation) is used to introduce the yfiK-containing plasmids into microorganisms which are then selected for plasmid-carrying clones by means of resistance to antibiotics, for example.

The invention therefore also relates to methods for preparing a microorganism strain of the invention, wherein a plasmid of the invention is introduced into a starting strain.

Production of amino acids of the phosphoglycerate family with the aid of a microorganism strain of the invention is carried out in a fermenter according to methods known per se.

The invention therefore also relates to a method for producing amino acids of the phosphoglycerate family, which comprises using a microorganism strain of the invention in a fermentation and removing the amino acid produced from the fermentation mixture.

The microorganism strain is grown in the fermenter as continuous culture, as batch culture or, preferably, as fed-batch culture. Particular preference is given to metering in a carbon source during fermentation.

Suitable carbon sources are preferably sugars, sugar alcohols or organic acids. Particular preference is given to using in the method of the invention glucose, lactose or glycerol as carbon sources.

Preference is given to metering in the carbon source in a form which ensures that the carbon source content in the fermenter is kept within a range from 0.1-50 g/l during fermentation. Particular preference is given to a range from 0.5-10 g/l.

Preferred nitrogen sources used in the method of the invention are ammonia, ammonium salts or proteinhydrolyzates. When using ammonia for correcting the pH stat, this nitrogen source continues to be metered in regular intervals during fermentation.

Further media additives which may be added are salts of the elements phosphorus, chlorine, sodium, magnesium, nitrogen, potassium, calcium, iron and, in traces (i.e. in μM concentrations), salts of the elements molybdenum, boron, cobalt, manganese, zinc and nickel.

It is furthermore possible to add organic acids (e.g. acetic acid, citric acid), amino acids (e.g. isoleucine) and vitamins (e.g. B1, B6) to the medium.

Complex nutrient sources which may be used are, for example, yeast extract, corn steep liquor, soybean meal or malt extract.

The incubation temperature for mesophilic microorganisms is preferably 15-45° C., particularly preferably 30-37° C.

The fermentation is preferably carried out under aerobic growth conditions. Oxygen is introduced into the fermenter by means of compressed air or by means of pure oxygen.

During fermentation, the pH of the fermentation medium is preferably in the range from 5.0 to 8.5, particular preference being given to pH 7.0. If production according to the invention of O-acetyl-L-serine is desired, the particularly preferred pH range is between 5.5 and 6.5.

Production of L-cysteine and L-cysteine derivatives requires feeding in a sulfur source during fermentation. Preference is given here to using sulfate or thiosulfate.

Microorganisms fermented according to the method described secrete in a batch or fed-batch process, after a growing phase, amino acids of the phosphoglycerate family into the culture medium with high efficiency over a period of from 10 to 150 hours.

### BRIEF DESCRIPTION OF THE DRAWINGS

Other objects and features of the present invention will become apparent from the following detailed description considered in connection with the accompanying drawing. It should be understood, however, that the drawing is designed for the purpose of illustration only and not as a definition of the limits of the invention.

In the drawing, wherein similar reference characters denote similar elements throughout the several views:

FIG. 1 shows the vector p G 13.

### DETAILED DESCRIPTION OF PREFERRED EMBODIMENTS

The following examples serve to further illustrate the invention.

### EXAMPLE 1

### Cloning of the yfiK Gene

The yfiK gene from Escherichia coli strain W3110 was amplified with the aid of polymerase chain reaction. The specific primers used were the oligonucleotides

yfiK-fw:

5′- (SEQ. ID. NO: 3) - 3′

and

yfik-rev:

5′- (SEQ. ID. NO: 4) - 3′.

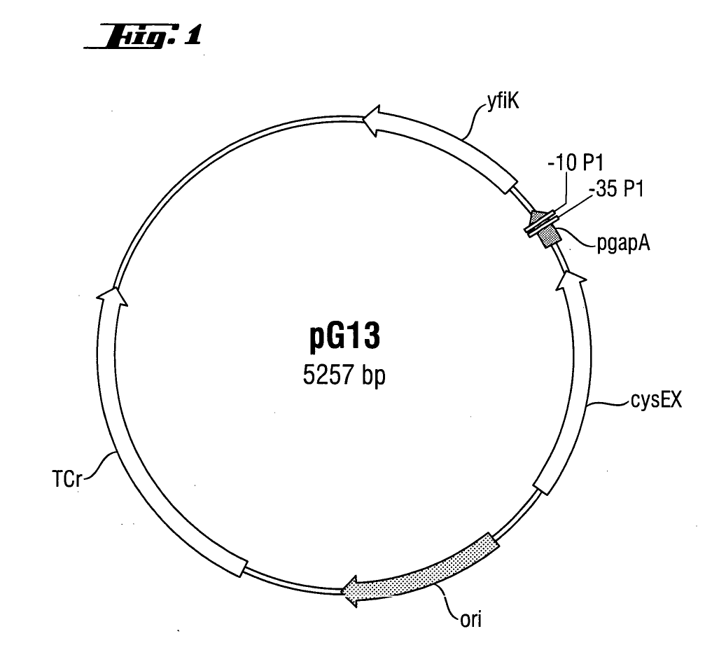

The resulting DNA fragment was digested by the restriction enzymes AsnI and PacI, purified with the aid of agarose gel electrophoresis and isolated (Qiaquick Gel Extraction Kit, Qiagen, Hilden, D). Cloning was carried out by way of ligation with an NdeI/PacI-cut vector pACYC184-cysEX-GAPDH which has been described in detail in EP0885962A1. This vector contains a cysEX gene coding for a serine acetyl transferase with reduced feedback inhibition by L-cysteine and, 3 thereof, the constitutive GAPDH promoter of the gapA gene. Said procedure places the yfiK gene downstream of the GAPDH promoter in such a way that transcription can be initiated therefrom. The resulting vector is referred to as pG13 and is depicted in FIG. 1 in the form of an overview drawing. Verification of the construct was followed by transforming Escherichia coli strain W3110 and selecting appropriate transformants using tetracycline. The bacteria strain Escherichia coli W3110/pG13 was deposited with the DSMZ (Deutsche Sammlung für Mikroorganismen und Zellkulturen GmbH, D-38142 Braunschweig) under the number DSM 15095 in accordance with the Budapest Treaty, and is utilized in the examples below as producer strain for producing amino acids of the phosphoglycerate family. The comparative strain chosen for demonstrating the effect of increased expression of the yfiK gene was W3110/pACYC184-cysEX which is likewise described in detail in EP0885962A1 but which contains, in contrast to pG13, no GAPDH promoter-yfiK sequence.

### EXAMPLE 2

### Producer Strain Preculture

A preculture for the fermentation was prepared by inoculating 20 ml of LB medium (10 g/l tryptone, 5 g/l yeast extract, 10 g/l NaCl), which additionally contained 15 mg/l tetracycline, with the strain W3110/pG13 or W3110/pACYC184-cysEX and incubation in a shaker at 150 rpm and 30° C. After seven hours, the entire mixture was transferred into 100 ml of SM1 medium (12 g/l K₂HPO₄; 3 g/l KH₂PO₄; 5 g/l (NH₄)₂SO₄; 0.3 g/l MgSO₄×7 H₂O; 0.015 g/l CaC₂×2 H₂O; 0.002 g/l FeSO₄×7 H₂O; 1 g/l Na₃citrate×2 H₂O; 0.1 g/l NaCl; 1 ml/l trace element solution comprising 0.15 g/l Na₂MoO₄×2 H₂O; 2.5 g/l Na₃BO₃; 0.7 g/l CoCl₂×6 H₂O; 0.25 g/l CuSO₄×5 H₂O; 1.6 g/l MnCl₃×4 H₂O; 0.3 g/l ZnSO₄×7 H₂O), supplemented with 5 g/l glucose, 0.5 mg/l vitamin B₁ and 15 mg/l tetracycline. Further incubation was carried out at 30° C. and 150 rpm for 17 hours.

### EXAMPLE 3

### Fermentative production of O-acetyl-L-serine

The fermenter used was a Biostat M instrument from Braun Biotech (Melsungen, D), which has a maximum culture volume of 2 1. The fermenter containing 900 ml of SM1 medium supplemented with 15 g/l glucose, 0.1 g/l tryptone, 0.05 g/l yeast extract, 0.5 mg/l vitamin B₁ and 15 mg/l tetracycline was inoculated with the preculture described in example 2 (optical density at 600 nm: approx. 3). During fermentation, the temperature was adjusted to 32° C. and the pH was kept constant at 6.0 by metering in 25% ammonia. The culture was gassed with sterilized compressed air at 1.5 vol/vol/min and stirred at a rotational speed of 200 rpm. After oxygen saturation had decreased to a value of 50%, the rotational speed was increased to up to 1 200 rpm via a control device in order to maintain 50% oxygen saturation (determined by a pO₂ probe calibrated to 100% saturation at 900 rpm). As soon as the glucose content in the fermenter had fallen from initially 15 g/l to approx. 5-10 g/l, a 56% glucose solution was metered in, feeding took place at a flow rate of 6-12 ml/h and the glucose concentration in the fermenter was kept constant between 0.5-10 g/l. Glucose was determined using the glucose analyzer from YSI (Yellow Springs, Ohio, USA). The fermentation time was 28 hours, after which samples were taken and the cells were removed from the culture medium by centrifugation. The resulting culture supernatants were analyzed by reversed phase HPLC on a LUNA 5 μ C18(2) column (Phenomenex, Aschaffenburg, Germany) at a flow rate of 0.5 ml/min. The eluent used was diluted phosphoric acid (0.1 ml of conc. phosphoric acid/l). Table 1 shows the contents obtained of the major metabolic product in the culture supernatant. Said products are O-acetyl-L-serine and N-acetyl-L-serine which is increasingly produced by isomerization from O-acetyl-L-serine under neutral to alkaline conditions.

TABLE 1

| | Amino acid content [g/l] | |
|---|---|---|
| Strain | O-acetyl-L-serine | N-acetyl-L-serine |
| W3110/pACYC184-cysEX | 1.8 | 1.5 |
| W3110/pG13 (cysEX-yfiK) | 7.4 | 3 |

### EXAMPLE 4

### Fermentative Production of N-acetyl-L-serine

N-Acetyl-L-serine was produced exactly as described in examples 2 and 3, merely adjusting the pH in the fermentation to 7.0. This facilitates isomerization of O-acetyl-L-serine to N-acetyl-L-serine and the major product obtained is N-acetyl-L-serine. The fermentation time was 48 hours.

TABLE 2

| | Amino acid content [g/l] |
|---|---|
| Strain | N-acetyl-L-serine |
| W3110/pACYC184-cysEX | 5.8 |
| W3110/pG13 (cysEX-yfiK) | 9.2 |

### EXAMPLE 5

### Fermentative Production of L-cysteine and L-cysteine Derivatives

L-Cysteine was produced exactly as described in examples 2 and 3, merely adjusting the pH in the fermentation to 7.0 and feeding in thiosulfate. The latter was fed in after two hours in the form of a 30% Na thiosulfate solution at a rate of 3 ml/h. The fermentation time was 48 hours. L-Cysteine production was monitored calorimetrically using the assay of Gaitonde (Gaitonde, M. K. (1967), Biochem. J. 104, 627-633). It has to be taken into account here that said assay does not discriminate between L-cysteine and the condensation product of L-cysteine and pyruvate (2-methylthiazolidine-2,4-dicarboxylic acid) described in EP 0885962 A1. LL-cystine which is produced from L-cysteine by oxidation is likewise detected as L-cysteine in the assay via reduction with dithiothreitol (DTT) in diluted solution at pH 8.0.

TABLE 3

| | Amino acid content [g/l] |
|---|---|
| Strain | L-cysteine + derivatives |
| W3110/pACYC184-cysEX | 4.6 |
| W3110/pG13 (cysEX-yfiK) | 7.5 |

According, while a few embodiments of the present invention have been shown and described, it is to be understood that many changes and modifications may be made thereunto without departing from the spirit and scope of the invention as defined in the appended claims.

What is claimed is:

1. A microorganism strain suitable for fermentative production of amino acids of the phosphoglycerate family or derivatives thereof and producible from a starting strain, having an increased activity of a yfiK-gene product or having an increased activity of a gene product of a yfiK homologue.

2. The microorganism strain as claimed in claim 1, which is selected from the group consisting of a fungus, a yeast, a bacterium, a member of the family Enterobacteriaceae, and a member of the species Escherichia coli.

3. The microorganism strain as claimed in claim 1, which is selected from the group consisting of a copy number of the yfiK gene being increased, and in which expression of said yfiK gene was increased by using suitable promoters or translation signals.

4. The microorganism strain as claimed in claim 3,
wherein a promoter is selected from the group consisting of constitutive GAPDH promoter of the gapA gene, inducible lac, tac, trc, lambda, ara and tet promoters.

5. The microorganism strain as claimed in claim 1,
which is an Escherichia coli strain in which the increased activity of a yfiK-gene product is based on an increase in a copy number of the yfiK gene in a pACYC derivative.

6. A plasmid, which comprises a yfiK gene with a promoter.

7. The plasmid as claimed in claim 6,
which additionally contains a genetic element for deregulation of cysteine metabolism.

8. A method for preparing a microorganism strain which comprises
introducing a plasmid as claimed in claim 6 into a starting strain.

9. A method for preparing an amino acid of the phosphoglycerate family, which comprises
using a microorganism strain as claimed in claim 1 in a fermentation mixture; and
removing the amino acid produced from the fermentation mixture.

10. The method as claimed in claim 9,
wherein the microorganism strain is grown in a fermenter as a culture selected from the group consisting of a continuous culture, a batch culture, and a fed-batch culture.

11. The method as claimed in claim 9,
wherein a carbon source is continuously metered in during fermentation.

12. The method as claimed in claim 11,
wherein the carbon source used is selected from the group consisting of a sugar, a sugar alcohol and an organic acid.

13. The method as claimed in claim 11,
wherein the carbon source is metered in,in a way so as to ensure that a content of the carbon source in a fermenter is kept in a range from 0.1-50 g/l, during fermentation.

14. The method as claimed in claim 13,
wherein the carbon source in the fermenter is kept in a range from 0.5-10 g/l, during fermentation.

15. The method as claimed in claim 9, 1wherein a nitrogen source is used and is selected from the group consisting of ammonia, an ammonium salt and a protein hydrolysate.

16. The method as claimed in claim 9,
wherein fermentation is carried out under aerobic growth conditions.


SEQUENCE LISTING (4 sequences)

SEQ ID NO: 1 - DNA, 750 bp, Escherichia coli
  CDS (110)..(694)
gatccataaccccaaacctatcgaaaatatcgaatctagaatataaaaacattcatttttttaaatgttccgtgtcgggtactgtctaccaaaacagaggagataacaagtgacaccgacccttttaagtgctttttggacttacaccctgattaccgctatgacgccaggaccgaacaatattctcgcccttagctctgctacgtcgcatggatttcgtcaaagtacccgcgtgctggcagggatgagtctgggatttttgattgtgatgttactgtgtgcgggcatttcattttcactggcagtgattgacccggcagcggtacaccttttgagttgggcgggggcggcatatattgtctggctggcgtggaaaatcgccaccagcccaacaaaggaagacggacttcaggcaaaaccaatcagcttttgggccagctttgctttgcagtttgtgaacgtcaaaatcattttgtacggtgttacggcactgtcgacgtttgttctgccgcaaacacaggcgttaagctgggtagttggcgtcagcgttttgctggcgatgattgggacgtttggcaatgtgtgctgggcgctggcggggcatctgtttcagcgattgtttcgccagtatggtcgccagttaaatatcgtgcttgccctgttgctggtctattgcgcggtacgcattttctattaacgaaaaaaagcggaagaggtcgccctcttccgcttagtaacttgctacttaag

SEQ ID NO: 2 - protein, 195 aa, Escherichia coli
VTPTLLSAFWTYTLITAMTPGPNNILALSSATSHGFRQSTRVLAGMSLGFLIVMLLCAGISFSLAVIDPAAVHLLSWAGAAYIVWLAWKIATSPTKEDGLQAKPISFWASFALQFVNVKIILYGVTALSTFVLPQTQALSWVVGVSVLLAMIGTFGNVCWALAGHLFQRLFRQYGRQLNIVLALLLVYCAVRIFY

SEQ ID NO: 3 - DNA, 35 bp, Artificial Sequence
  Primer for PCR
ggaattcattaatgatccataaccccaaacctatc

SEQ ID NO: 4 - DNA, 33 bp, Artificial Sequence
  Primer for PCR
gccttaattaagtagcaagttactaagcggaag